(19) United States
(12) Patent Application Publication
(10) Pub. No.: US 2021/0244558 A1
(43) Pub. Date: Aug. 12, 2021

(54) COUNTERFORCE BRACE

(71) Applicant: Gordon Consulting, Inc., Kansas City, MO (US)

(72) Inventor: Howard A. Gordon, Kansas City, MO (US)

(21) Appl. No.: 16/789,048

(22) Filed: Feb. 12, 2020

(51) Int. Cl. A61F 5/01

(52) U.S. Cl. CPC A61F 5/0111

(57) ABSTRACT

Provided herein are a foot bracing device and methods of using the device to anatomically stabilize the three primary aspects of foot movement desired to protect the ankle complex during load bearing forces.

### FIELD OF THE INVENTION

The present invention relates generally to a stabilizing foot bracing device.

### BACKGROUND OF THE INVENTION

The supination of a foot combines inward rotation at the ankle, adduction of the hindfoot, inversion of the forefoot, and medial arch elevation. In short, supination produces a medial (inward) movement at the talocalcaneal (sub talus) joint known as inversion as well as similar movement between the hindfoot (talus) and midfoot (navicular) bones.

A foot that supinates appears to naturally favor the lateral region or outside edge of the foot. A certain amount of supination is normal during the toe-off stage of running, i.e., when the heel first lifts off the ground until the end of the step. During excessive supination, the lateral structures of the foot tighten which may rupture or avulse portions of the lateral collateral ligaments resulting in a “rolling over” of the ankle or ankle sprain.

The pronation of a foot produces opposite foot behavior of the subtalar joint, including adduction of the forefoot, eversion of the hindfoot and dorsiflexion. Pronation produces a lateral (outward) movement of the foot at the subtalar joint known as eversion as well as similar hindfoot and midfoot movements. A foot that pronates appears to favor the inside or medial edge of the foot naturally. The foot naturally pronates to absorb the shock resulting from the heel hitting the ground. Excessive pronation increases the stress on the inside/medial portion of the foot which may lead to various injuries such as arch strain, heel pain, inner ankle pain and shin splints.

Accordingly, it is desirable to preclude excessive pronation and/or supination of the ankle joint and foot. Thus, various devices have been proposed to prevent ankle and foot injuries by attempting to maintain the foot in a “neutral position.” Although assumedly effective in their operation, such devices have failed to simultaneously provide effective stabilization to the forefoot, transverse arch, and ankle complex for the safety of the foot from heel-strike to toe-off thereby providing proper anatomical foot movement during walking, running or jumping movements.

There is a need for a device that provides proper anatomical foot movement during walking, running, or jumping movements by simultaneously stabilizing the forefoot, transverse arch, and ankle complex for safety of the foot from heel-strike to toe-off.

### BRIEF DESCRIPTION OF THE DRAWINGS

FIG. 1 is a diagrammatic view showing pronation of the right foot from the rear.

FIG. 2 is a diagrammatic view showing supination of the right foot from the rear.

FIG. 3 is a top/dorsal view of the right foot of the wearer. Metatarsals and phalanges of the foot of the wearer and the second edge of the body are shown in phantom lines.

FIG. 4 is a perspective view of the medial side of the right foot of the wearer in the brace. The medial and metatarsal straps and the circumferential strap, partially shown behind the brace, are disengaged.

FIG. 5 is a perspective view of the medial side of the right foot of the wearer in the brace. The circumferential strap is engaged around the brace.

FIG. 6 is a perspective view of the medial side of the right foot of the wearer in the brace. The circumferential strap is engaged around the brace. The medial strap is extended across the dorsal aspect of the forefoot, rearwardly underneath the arch of the wearer towards the lateral side of the heel, and upwardly along the medial side of the ankle joint region.

FIG. 7 is a perspective view of the lateral side of the right foot of the wearer in the brace. The circumferential strap is engaged around the brace. The medial strap is extended across the dorsal aspect of the forefoot.

FIG. 8 is a perspective front view of the right foot of the wearer in the brace. The circumferential strap is engaged around the brace. The medial and lateral straps are wrapped and secured around the foot.

FIG. 9 is a bottom view of the right foot of the wearer in the brace. The medial strap is shown extending from the second edge of the brace underneath the arch of the wearer towards the lateral side of the heel. The lateral strap is shown extending from the second edge of the brace underneath the arch of the wearer towards the medial side of the heel.

FIG. 10 is a lateral view of the foot of the wearer secured in the brace.

FIG. 11 is a bottom view of the right foot of the wearer in the brace showing the lateral and medial straps attached to the second edge.

FIG. 12 is a dorsal view of the right foot of the wearer in the brace. The medial and lateral straps are disengaged. Arrows depict the direction of extension of the lateral and medial straps around the foot.

FIG. 13 is a side view of the lateral side of the right foot of the wearer in the brace. The circumferential strap is engaged around the brace. The medial and lateral straps are wrapped around the foot, drawn upwards and attached to the complementary means of attachment on the sides of the brace above the ankle joint.

FIG. 14 is a bottom view of the right foot of the wearer in the brace. The medial and lateral straps are disengaged and indicate the means for attaching the straps to the brace above the ankle joint.

FIG. 15 is a rear view of the right foot of the wearer with the brace secured thereon.

FIG. 16 is a side view of the lateral side of the right foot of the wearer in the brace. The circumferential strap is positioned to engage around the brace. The medial is disengaged.

FIG. 17 is a view of the pattern of the circumferential strap.

FIG. 18 is a view of the pattern of the first integral sleeve.

FIG. 19 is a view of the pattern of the second integral sleeve.

FIG. 20 is a view of the pattern of the foot plate.

FIG. 21 is a view of the pattern of the lateral and medial countervailing force straps.

FIG. 22 is a view of the pattern of the lateral and medial heel lock and torque strap.

### DETAILED DESCRIPTION

The present disclosure concerns a brace incorporating a bi-lateral strapping mechanism engineered to anatomically stabilize the three primary aspects of foot movement desired to protect the ankle complex during load-bearing forces. The brace and methods of using the brace are described below.

### I. Brace

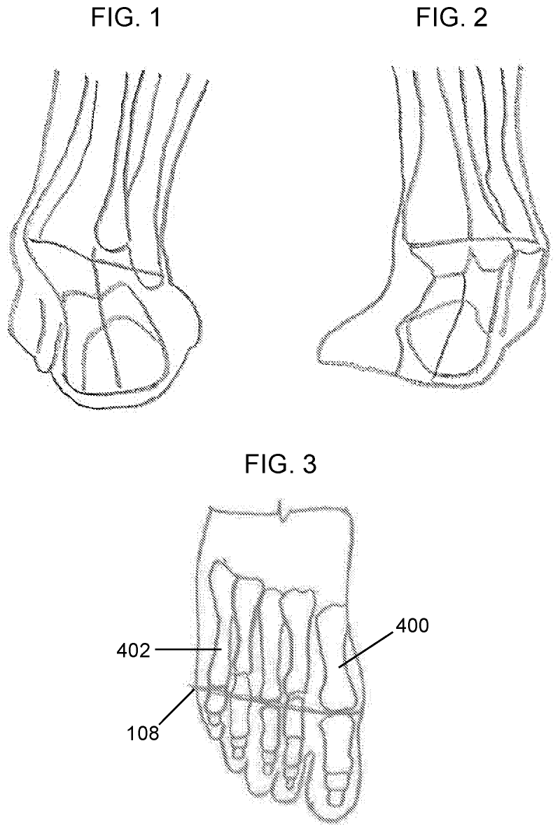

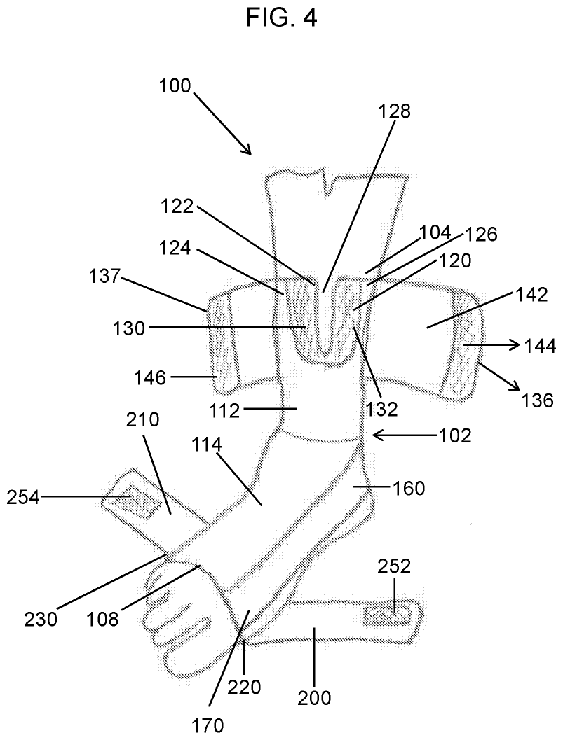

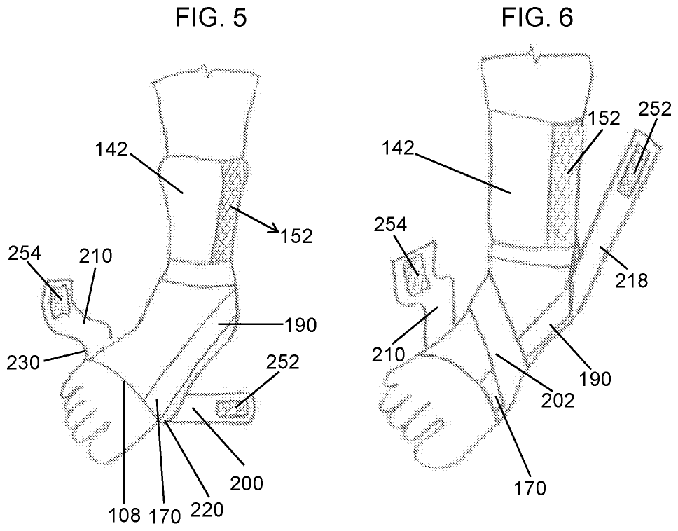

The brace incorporates a bi-lateral strapping mechanism engineered to anatomically stabilize the three primary aspects of foot movement desired to protect the ankle complex during load-bearing forces. More specifically, the bi-lateral straps are anatomically lengthened, positioned and attached to the body of the device at the respective distal ends of the first and fifth metatarsals to affect the following ankle complex securing results. Initially, each strap will stabilize the forefoot from excess inversion/eversion movement, thereby commence securing the forefoot from unwarranted rolling. Next, said straps crisscross over the dorsal aspect of the foot, securely, flowing under the transverse arch to reduce excess pronation/supination (FIGS. 1 and 2) at mid-stance, a second anatomical location to control unwarranted medial or lateral rolling of the foot, specifically protecting at least the Anterior Talofibular Ligament, the Calcaneofibular Ligament, the Posterior Talofibular Ligament, and the Posterior Tibiofibular Ligament. Each strap then extends to bi-laterally grasp the heel, then each flow upward to securely attach to their respective medial/lateral upper aspects of the brace. The brace and methods of using the brace are described below.

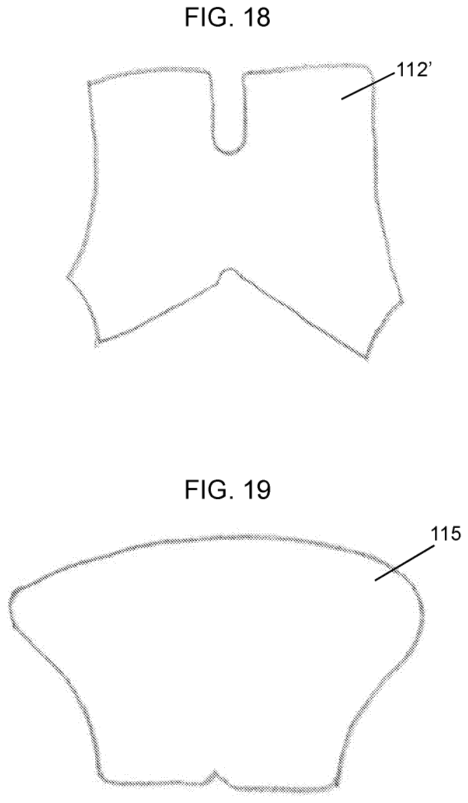

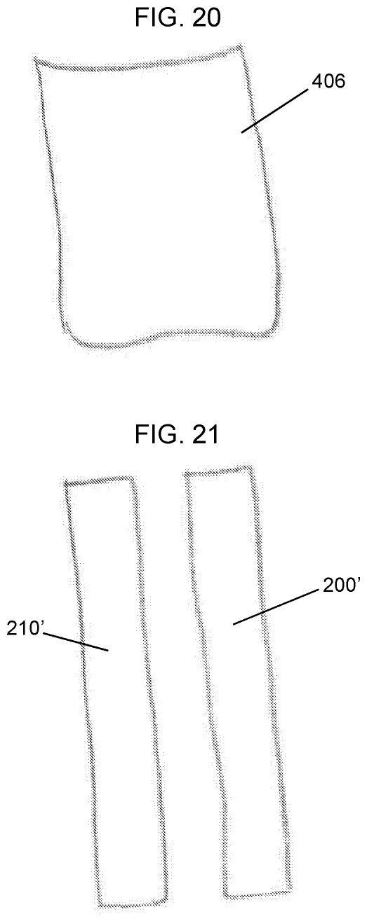

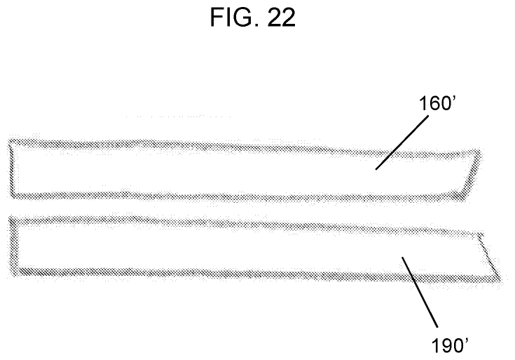

Turning more particularly to the drawings, brace 100 comprises a sock-like body 102. The body 102 provides a compression fit about the ankle joint complex and forefoot of the wearer. The body 102 provides a compression fit about the ankle joint complex of the wearer and extending to a compression fit surrounding the forefoot of the wearer. In some aspects, the body 102 comprises a first integral sleeve 112 providing a compression fit at and above the ankle joint complex of the wearer and a second integral sleeve 114 providing a compression fit on the forefoot of the wearer. The forefoot is described as that region of the foot extending from the distal/head ends of the first 400 and fifth metatarsals 402 through the phalanges (FIG. 1). The body 102 comprises a first free edge 104 circumscribing a first aperture operable to encompass the foot at a superior location above the ankle joint complex. The body 102 further comprises a second free edge 108 circumscribing a second aperture operable to encompass the foot adjacent the distal ends/heads of the first 400 and fifth 402 metatarsals. In some aspects, free edge 108 does not extend beyond the distal ends/heads of the first 400 and fifth 402 metatarsals as it may reduce dorsal flexion in the foot. A such, upon slipping the body 102 over the foot, the body 102 presents a compression fit about the ankle joint complex and forefoot of the wearer between the first edge 104 and the second edge 108. In some aspects, body 102 can further comprise an aperture 116 at the heel to allow for extension of the heel therethrough (shown in FIGS. 8, 14, and 18). When the body 102 comprises a first integral sleeve 112 connected to a second integral sleeve 114, the first integral sleeve 112 can be constructed from pattern 112′, and the second integral sleeve 114 can be constructed from dorsal pattern 115 and foot plate pattern 406.

The initial extension of the foot through the first aperture presented by free edge 104 can be enhanced by a closure system 120 (FIG. 2). The closure system 120 comprises a notch 122, which extends from the free edge 104 of sleeve 112 and partially bisects integral sleeve 112 between flaps 124 and 126. In some aspects, the notch 102 is U-shaped, although other shapes can also be appropriate and will be recognized by individuals of skill in the art. As such, the closure system 120 allows the user to increase the initial opening presented by the upper free edge 104 such that feet of various sizes may be initially extended through this free edge 104 for ultimate extension of the phalanges 404 through the downstream free edge 108. The closure system 120 comprises means for fastening flap 124 to flap 126 to secure the free edge 104 around the foot of the wearer above the ankle joint complex. The closure system 120 enables the user to adjust the compression of the sleeve 112 about the ankle joint region as the degree of overlap of flaps 124 and 126 can be selectively adjusted.

In some aspects shown in the figures herein, the means for fastening flap 124 to flap 126 is a loop and hook system, such as Velcro™ fastening material. However, one of ordinary skill in the art will appreciate that other means of fastening may be used, including, but not limited to, buttons, snaps, zippers, buckles, semi-permanent adhesives, or any combination of the above. As shown in the aspect depicted in the figures, the fastening system can comprise tab 130 which is secured along the longitudinal extent of the notch 122 on flap 124, and tab 132 which is secured along the longitudinal extent of the notch 122 on flap 126. Tabs 130 and 132 have complementary hook and loop fastener material attached thereon for securing flap 124 to flap 126. In some aspects, a tongue 128 is attached to the interior of sleeve 112 for extension along notch 122 so as to preclude the flaps 124 and 126, and tabs 130 and 132 of the closure system 120 from directly contacting the user.

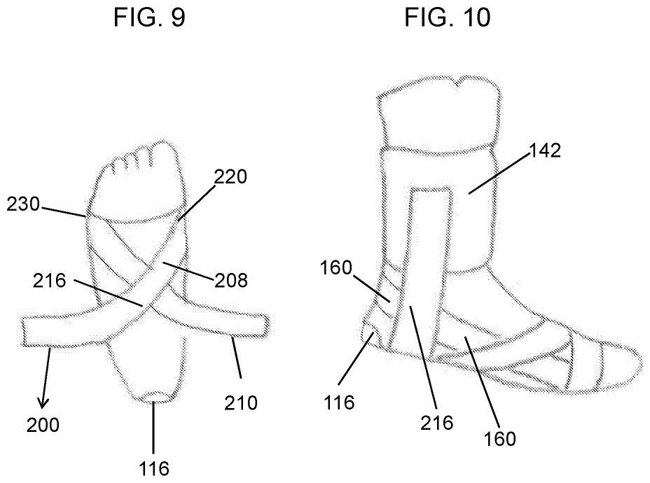

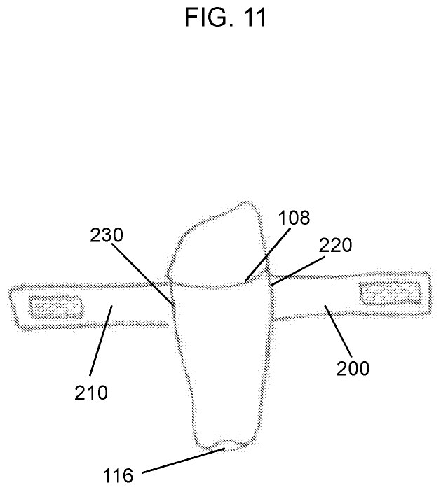

The brace 100 further comprises elongated medial 200 and lateral 210 countervailing force straps which are anchored at a location 220 and 230, respectively, at the lateral and medial edge of the sleeve 114 and adjacent the free edge 108 of the sleeve 114 (FIGS. 9, 14, and 17). It is understood that the straps 200 and 210 may be attached together to form a single elongated strap anchored at locations 220 and 230 or any location therebetween so as to form a single strap with the medial 200 and lateral 210 straps or separate straps extending therefrom. The end 204 of medial strap 200 comprises a means of attaching 252 the end 204 to a first complementary means of attachment on the medial side of brace 100 at a position on the medial side of the brace about the ankle joint. Further, the end 205 of lateral strap 210 comprises a means of attaching 254 the end 205 to a second complementary means of attachment on the brace at a position on the medial side of the brace about the ankle joint. Some aspects of this invention may utilize four or more straps to provide the countervailing forces necessary to prevent excessive supination or pronation of the foot and ankle joint and/or to provide compressive forces on the ankle joint. In some aspects, the medial 200 and lateral 210 countervailing force straps are elastic straps. In other aspects, the medial 200 and lateral 210 countervailing force straps are not elastic. The medial 200 and lateral 210 countervailing force straps can be made from patterns 200′ and 210′, respectively.

As such, a first extension 202 of medial strap 200 extends from anchor location 210 to the medial side of the foot, across the dorsal aspect of the foot and rearwardly towards the superior aspect of the heel (FIG. 6). A second extension 208 of the strap 200 extends underneath the arch as best shown in FIG. 9. A third extension 218 of the remaining portion of the strap 200 extends upwardly along the medial side of the ankle joint region as best shown in FIG. 6. The attachment means 252 at end 204 of medial strap 200 is attached to a first complementary means of attachment on the brace 100 at a position on the medial side of the brace about the ankle joint. The attachment means can be as described above. In some aspects, the complementary attachment means are complementary hook and loop attachment means. Accordingly, the right foot being shown, the medial strap 200 is wrapped around the foot of the wearer so as to provide a force to secure the forefoot, heel and ankle regions of the wearer.

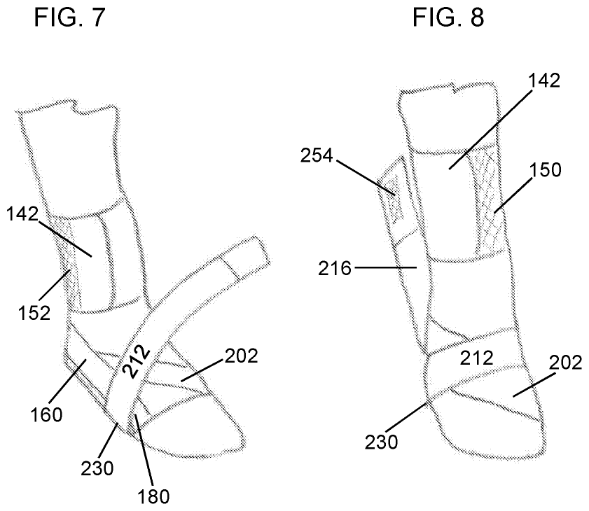

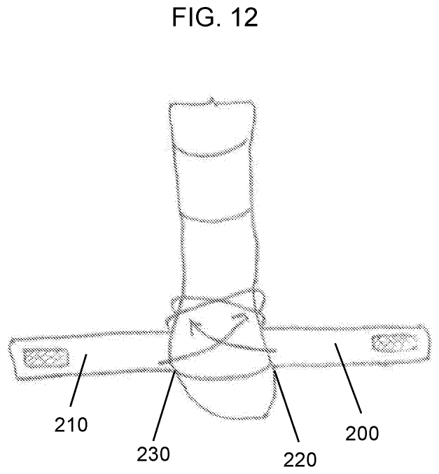

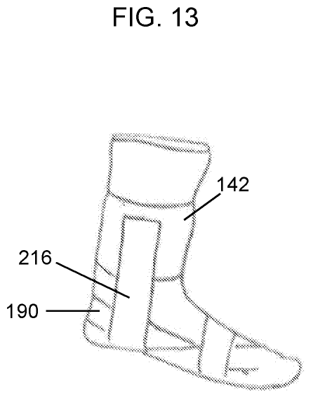

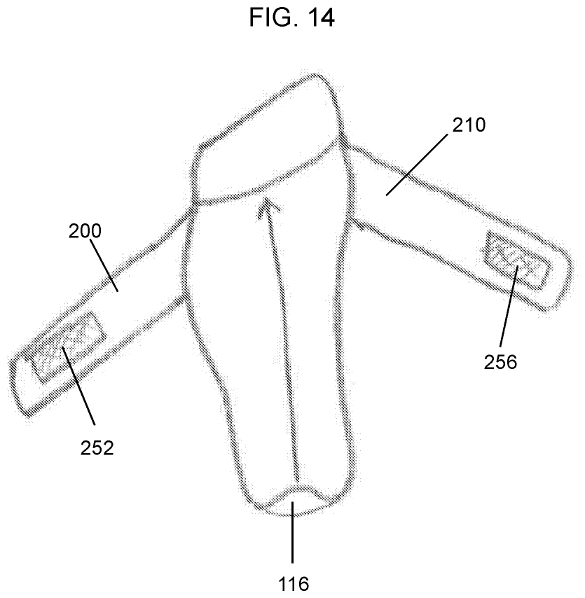

Continuing with the figures as above, a first extension 212 of lateral strap 210 extends from anchor location 230 above the dorsal aspect of the forefoot and rearwardly towards the medial side of the heel (FIGS. 7, 8 and 12). A second strap extension 214 is then wound underneath the arch (FIG. 9) with a third extension 216 brought upwardly along the lateral side of the heel and ankle joint (FIG. 10). The attachment means 254 at end 205 of lateral strap 210 is attached to a second complementary means of attachment on brace 100 at a position on the medial side of the brace about the ankle joint. The attachment means can be as described above. In some aspects, the complementary attachment means are complementary hook and loop attachment means. Accordingly, the right foot being shown, the lateral strap 210 is wrapped just prior to the forefoot of the wearer so as to provide a stabilizing force to the forefoot, heel and ankle regions of the wearer.

In some aspects, the first and second means for attachment on the medial and lateral sides of the brace, respectively, can be a first means of attachment (not shown) on the medial side of the body 102 and a second means of attachment (not shown) on the lateral side of the body 102. As explained above, the first means of attachment is complementary to attachment means 252 at end 204 of medial strap 200, and the second means of attachment is complementary to attachment means 254 at end 205 of medial strap 210.

In some aspects, the brace further comprises a circumferential strap 142 for wrapping around the first sleeve 112 about the ankle joint complex of the foot of the wearer. The strap 142 is made from pattern 142′ and is generally rectangular in shape, although other appropriate shapes can be envisioned. For instance, the strap can have a shape configured to follow the contours of a foot about the ankle complex. The strap 142 comprises a left edge 136, and a right edge 137. The strap 142 comprises a closure system 140 for securing the strap around the first sleeve 112 above the ankle joint complex of the foot of the wearer adjacent edge 104. The closure system 140 comprises means for fastening the left edge 136 to the right edge 137 to secure the strap 142 around sleeve 112 and the foot. The fastening of the left edge 136 to the right edge 137 enables the user to adjust the compression of the strap 142 about the ankle joint region as the degree of overlap of edges 136 and 137 can be selectively adjusted. The degree of overlap of the left edge 136 to the right edge 137 adjusts the compressive fit of strap 142, about the ankle joint and thus maintains the circumferential integrity of the brace about the ankle joint complex. In some aspects shown in the figures herein, the means for fastening edges 136 and 137 is a loop and hook system, such as Velcro™ fastening material. However, one of ordinary skill in the art will appreciate that other means of fastening may be used, including, but not limited to, buttons, snaps, zippers, buckles, semi-permanent adhesives, or any combination of the above. As shown in the aspect depicted in the figures, the fastening system can comprise a first tab 144 which is secured along the length of the left edge 136 of strap 142, and a second tab 146 which is secured along the length of the right edge 137 of strap 142. Tabs 144 and 146 have complementary hook and loop fastener material attached thereon for securing edges 136 and 137. Accordingly, the strap 142 is wrapped circumferentially about the ankle joint complex and edges 136 and 137 are attached one to the other. The strap 142 can be made from pattern 142′.

In some aspects, the first and second means for attachment on the medial and lateral sides of the brace, respectively, can be a first means of attachment 150 on the medial side of the strap 142 and a second means of attachment 152 on the lateral side of the strap 142. As explained above, the first means of attachment 150 is complementary to attachment means 252 at end 204 of medial strap 200, and the second means of attachment 152 is complementary to attachment means 254 at end 205 of medial strap 210

In some aspects, the strap 142 is permanently attached at the rear of body portion 112 adjacent edge 104. Alternatively, the strap 142 can be removably attached to the back of sleeve 112 using other means for attaching, including but not limited to, buttons, snaps, buckles, zippers, semi-permanent adhesives, or combinations of the above.

In some aspects, the brace 100 further comprises a lateral heel lock and torque strap 160 and a medial heel lock and torque strap 190 providing enhanced torque forces to control inversion/eversion movement of the forefoot, stabilizing torque force to the arch, and heel locking forces when attaching the bilateral straps 200 and 210 to the brace. The heel straps function as a stabilizing heel locking mechanism, in a manner similar to an ankle taping procedure. The lateral heel strap 160 comprises a first end 180 attached at a first attachment point at edge 108 on the medial side of the body 102 and a second end 182 attached at the rear of the body 102. The lateral heel strap 160 extends from the first attachment point 180 along the medial side of the foot around the heel to the second attachment point 182 at the rear of the body 102. The medial heel strap 190 comprises a first end 170 attached at a first attachment point at edge 108 on the medial side of the body 102 and a second end 172 attached at the rear of the body 102. The medial heel strap 190 extends from the first attachment point along the medial side of the foot to the second attachment point at the rear of the body 102. It is understood that the straps 160 and 190 may be attached together to form a single elongated strap attached at the rear of the body 102 or any location therebetween so as to form a single strap with the medial heel lock 190 and lateral heel lock 160 straps or separate straps extending therefrom. The lateral heel strap 160 can be made from pattern 160′ and the medial heel strap 190 can be made from pattern 190′.

In some aspects, the lateral heel strap 160 and the medial heel strap 190 are an integral part of the body 102 and are permanently attached to the body 102 along the length of strap 160 and strap 190. In other aspects, the lateral heel strap 160 and the medial heel strap 190 are permanently but partially attached at the rear of the body 102. In yet other aspects, lateral heel strap 160 and the medial heel strap 190 are removably attached to the body 102. When the lateral heel strap 160 and the medial heel strap 190 are removably or partially attached to the body 102, the heel strap 160 and the medial heel strap 190 further comprise means of attaching the straps 160 and 190 to the body 102. Means for removably attaching can be as described above.

Accordingly, the body 102 provides compressive forces about the ankle and foot of the user. The medial 200 and lateral 210 straps present countervailing forces beginning at the metatarsal heads, along the forefoot and upward along the ankle joint complex. As previously discussed, pronation and supination of the foot respectively present opposed movements at the subtalar joint, hindfoot, and midfoot such that the foot favors the lateral region foot (supination) or the medial region (pronation) of the foot. Straps 200 and 210 thus present countervailing forces in these regions to resist movement of the ankle and foot, inclusive of its underlying structures, from excessive pronation or supination. Thus, as the foot is held in a neutral position, the probability of the user landing on an excessively supinated or pronated foot during walking, jumping and/or running is reduced which reduces the probability or for potential degree of injury. The use of these counter force straps 200 and 210, with or without the compressive fit offered by the body 102 and the compression provided by closure system 120 and circumferential strap 142, enhances the prophylactic advantages of the lateral heel and torque strap 160 and the medial heel and torque strap 190.

Any material capable of providing a sufficiently compressive fit, strength, and comfort can be appropriate for constructing the sock like body 102 of the brace. In some aspects, the body 102 is constructed of PES Oxford fabric with PVC coating. Any material of sufficient strength can be appropriate for constructing the straps. In some aspects, straps are constructed of multiple strength polyester materials. In some aspects, the brace is constructed using sewn seams for structural strength.

### II. Methods

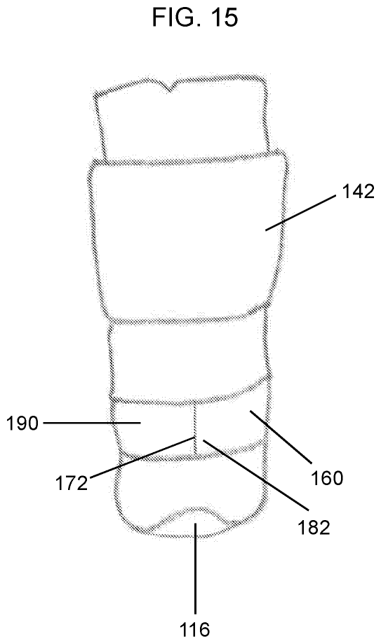

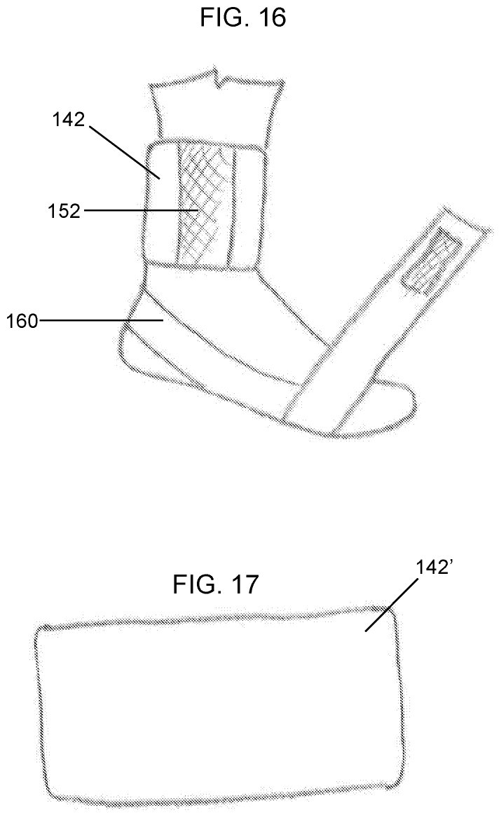

Further provided herein is a method of preventing the excessive supination or pronation of a user's foot and ankle joint by using the stabilizing brace 100. The method comprises having a wearer place their foot into the brace 100 first by placing their foot into the first opening 106 at the first edge 104 and extending the foot through the second opening 110 of the second edge 108. The wearer then wraps the medial strap 200 around the foot by extending the medial strap through a first extension 202 of strap 200 from anchor location 210 to the medial side of the foot, across the dorsal aspect of the forefoot and rearwardly towards the lateral side of the heel as best depicted in FIG. 15. The medial strap 200 is further wrapped by extending through a second extension 208 underneath the arch and through a third extension 218 of the remaining portion of the strap 200 upwardly along the medial side of the ankle joint region. The wearer then attaches end 204 of strap 200 to the first complementary means of attachment on the brace at a location on the medial side of the brace above the ankle joint.

The wearer then wraps the lateral strap 210 around the foot by extending the medial strap through a first extension 212 of strap 210 from anchor location 230 to the medial side of the foot, across the dorsal aspect of the forefoot and rearwardly towards the medial side of the heel. The lateral strap 210 is further wrapped by extending through a second extension 214 underneath the arch and through a third extension 216 of the remaining portion of the strap 210 upwardly along the lateral side of the ankle joint region. The wearer then attaches end 205 of strap 210 to the second complementary means of attachment on the brace at a location on the lateral side of the brace above the ankle joint.

In some embodiments of the method, when the brace further comprises the closure system 120, the wearer also manipulates the flaps 124, 126 of the notch 122 to adjust the size of the first opening 106 of the first edge 104 to tighten the fit of the sleeve 112 or to provide additional compressive forces about the ankle joint region.

In some aspects, when the brace further comprises the circumferential strap 142, the method further comprises securing the strap around the body by fastening the left edge to the right edge. In one aspect, when the first complementary means of attachment on the brace 100 is at a position on the medial side of the circumferential strap 142, and the second complementary means of attachment on the brace 100 is at a position on the lateral side of the circumferential strap 142, the method further comprises attaching the end 204 of the medial strap 200 to the first complementary means of attachment 150 on the circumferential strap 142 and attaching the end 205 of the lateral strap 210 to the second complementary means of attachment 152 on the circumferential strap.

In some aspects, when the brace further comprises the lateral heel strap 160 and the medial heel strap 190 and the straps 160 and 190 are permanently but partially attached at the rear of the body 102 or removably attached to the body 102, the method further comprises attaching the end 180 of the lateral strap 160 to the first attachment point at edge 108 on the lateral side of the body 102, and attaching the end 170 of the medial strap 160 to the second attachment point at edge 108 on the medial side of the body 102.

Although a now preferred embodiment of this invention has been described herein, it is understood that such a description is not limited except as set forth in the claims and allowable, functional equivalents thereof.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a,” “an,” “the” and “said” are intended to mean that there are one or more of the elements. The terms “comprising,” “including” and “having” are intended to be inclusive and mean that there may be additional elements other than the listed elements.

As various changes could be made in the above-described cells and methods without departing from the scope of the invention, it is intended that all matter contained in the above description and in the examples given below, shall be interpreted as illustrative and not in a limiting sense.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention.

1. A foot stabilizing brace comprising:
a. a body having a sock-like configuration operable to encompass an ankle joint and a portion of a wearer's forefoot, the body comprising:
i. a first free edge comprising a first aperture, the first free edge operable to encompass the ankle of the wearer at a superior location above the ankle joint;
ii. a second free edge comprising a second aperture, the second free edge operable to encompass the foot of the wearer approximate distal ends of metatarsals of the foot upon extension of the wearer's foot therethrough, wherein the body extends between the first and second free edges;
b. a medial strap comprising an elongated body comprising a first end and a second end, wherein the first end is anchored at a first location at the second free edge of the body, wherein the first anchor location is at the medial edge of the foot; the second end comprising a means of reversibly attaching the second end to a first complementary means of attachment on the brace at a position on the medial side of the brace above the ankle joint; and
c. a lateral strap comprising an elongated body comprising a first end and a second end, wherein the first end is anchored at a first location at the second free edge of the body, wherein the first anchor location is at the lateral edge of the foot; the second end comprising a means of reversibly attaching the second end to a second complementary means of attachment on the brace at a location on the lateral side of the brace above the ankle joint;
wherein the medial and lateral straps are operable to provide countervailing forces along the foot and ankle joint to resist excessive supination and pronation thereof.

2. The brace of claim 1, wherein the medial strap is operable to extend from the first anchored end to a lateral position on a wearer's arch, then underneath the wearer's arch to a medial side of the wearer's arch, then to a superior location above the ankle joint; and wherein the lateral strap is operable to extend from the second anchored end to a medial position on the wearer's arch, then underneath the wearer's arch to a lateral side of the wearer's arch, then to a superior location above the ankle joint.

3. The brace of claim 2,
wherein the medial strap comprises a first integral extension operable to medially extend from the first anchored end across a dorsal aspect of the wearer's foot to the lateral position on the wearer's arch; a second integral extension operable to extend from the lateral position on the wearer's arch, underneath the wearer's arch to the medial side of the brace; and a third integral extension operable to extend from the medial side of the wearer's arch to a superior location above the ankle joint; and
wherein the lateral strap further comprises a first integral extension operable to laterally extend from the second anchored end across the dorsal aspect of the foot to a medial position on the wearer's arch; a second integral extension operable to extend from the medial position on the wearer's arch, underneath the wearer's arch to a lateral side of the wearer's arch; and a third integral extension operable to extend from the lateral side of the wearer's arch to a superior location above the ankle joint.

4. The brace of claim 1, wherein the first complementary means of attachment on the brace is on the medial side of the body and wherein the second complementary means of attachment on the brace is on the lateral side of the body.

5. The brace of claim 1 further comprising a closure system comprising a notch in the body extending from the first free edge, the notch forming a first flap and a second flap in the body, means for fastening the first flap to the second flap to secure the first free edge around the foot of the wearer above the ankle joint complex, wherein manipulation of the first and second flaps adjusts a size of the first aperture.

6. The brace of claim 5 further comprising a tongue attached to the interior of the body and configured to extend along and span and to preclude the closure system from directly contacting the user spanning the notch.

7. The brace of claim 1, wherein the first complementary means of attachment on the brace is at a position on the medial side of the body above the ankle joint, and the second complementary means of attachment on the brace is at a position on the lateral side of the body above the ankle joint.

8. The brace of claim 1 further comprising a circumferential strap comprising a left edge and a right edge, a closure system for securing the strap around the body adjacent the first edge, wherein the closure system comprises means for fastening the left edge to the right edge to secure the strap around the body.

9. The brace of claim 8, wherein the first complementary means of attachment on the brace is at a position on the medial side of the circumferential strap, and the second complementary means of attachment on the brace is at a position on the lateral side of the circumferential strap.

10. The brace of claim 1, further comprising a heel torque strap comprising a first end and a second end, wherein the first end attaches at a first attachment point on the medial side of the body and the second end attaches at a second attachment point on the medial side of the body, and wherein the heel strap extends from the first attachment point along the medial side of the foot around the heel, then extends along the lateral side of the foot to the second attachment point.

11. A method of preventing excessive supination or pronation of an ankle, the method comprising:
a. providing a stabilizing brace, the brace comprising:
i. a body having a sock-like configuration operable to encompass an ankle joint and a portion of a wearer's forefoot, the body comprising:
1) a first free edge comprising a first aperture, the first free edge operable to encompass the ankle of the wearer at a superior location above the ankle joint,;
2) a second free edge comprising a second aperture, the second free edge operable to encompass the foot of the wearer approximate distal ends of metatarsals of the foot upon extension of the wearer's foot therethrough, wherein the body extends between the first and second free edges;
ii. a medial strap comprising an elongated body comprising a first end and a second end, wherein the first end is anchored at a first location at the second free edge of the body, wherein the first anchor location is at the medial edge of the foot; the second end comprising a means of reversibly attaching the second end to a first complementary means of attachment on the brace at a position on the medial side of the brace above the ankle joint; and
iii. a lateral strap comprising an elongated body comprising a first end and a second end, wherein the first end is anchored at a first location at the second free edge of the body, wherein the first anchor location is at the lateral edge of the foot; the second end comprising a means of reversibly attaching the second end to a second complementary means of attachment on the brace at a location on the lateral side of the brace above the ankle joint;
wherein the medial and lateral straps are operable to provide countervailing forces along the foot and ankle joint to resist excessive supination and pronation thereof.
b. extending the wearer's foot through the first aperture of the first edge and extending the foot through the second opening of the second edge;
c. wrapping the medial strap around the foot by extending the medial strap from the first anchored end to a lateral position on the wearer's arch, then underneath the wearer's arch to a medial side of the wearer's arch, then upwardly along the medial side of the ankle joint complex to a superior location above the ankle joint, and attaching the second end to the first complementary means of attachment on the brace at a location on the medial side of the brace above the ankle joint;
d. wrapping the lateral strap around the foot by extending the lateral strap from the first anchored end to a medial position on the wearer's arch, then underneath the wearer's arch to a lateral side of the wearer's arch, then upwardly along the lateral side of the ankle joint complex to a superior location above the ankle joint, and attaching the second end to the second complementary means of attachment on the brace at a location on the lateral side of the brace above the ankle joint.

12. The method of claim 11, further comprising extending the medial strap from the first anchored end to a lateral position on a wearer's arch, then underneath the wearer's arch to a medial side of the wearer's arch, then to a superior location above the ankle joint and attaching the second end to the first complementary means of attachment on the brace at a position on the medial side of the brace above the ankle joint; and extending the lateral strap from the first anchored end to a medial position on a wearer's arch, then underneath the wearer's arch to a lateral side of the wearer's arch, then to a superior location above the ankle joint and attaching the second end to the second complementary means of attachment on the brace at a position on the lateral side of the brace above the ankle joint.

13. The method of claim 11, wherein the brace further comprises a closure system comprising a notch in the body extending from the first free edge, the notch forming a first flap and a second flap in the body, means for fastening the first flap to the second flap to secure the first free edge around the foot of the wearer above the ankle joint complex, and the method further comprises manipulating the first and second flaps to adjust a size of the first aperture.

14. The method of claim 11, wherein the first complementary means of attachment on the brace is on the body at a position on the medial side of the body above the ankle joint, the second complementary means of attachment on the brace is on the body at a position on the lateral side of the body above the ankle joint, and wherein the method further comprises attaching the second end of the medial strap to the first complementary means of attachment on the body and attaching the second end of the lateral strap to the second complementary means of attachment on the body.

15. The method of claim 11, wherein the brace further comprises a circumferential strap comprising a left edge and a right edge, a closure system for securing the strap around the body adjacent the first edge of the body, wherein the closure system comprises means for fastening the left edge to the right edge to secure the strap around the body, and wherein the method further comprises securing the strap around the body by fastening the left edge to the right edge.

16. The method of claim 15, wherein the first complementary means of attachment on the brace is at a position on the medial side of the circumferential strap, and the second complementary means of attachment on the brace is at a position on the lateral side of the circumferential strap, and wherein the method further comprises attaching the second end of the medial strap to the first complementary means of attachment on the circumferential strap and attaching the second end of the lateral strap to the second complementary means of attachment on the circumferential strap.
